(19) United States
(12) Patent Application Publication
(10) Pub. No.: US 2026/0115244 A1
(43) Pub. Date: Apr. 30, 2026

(54) INJECTABLE NUTRIENT SOLUTION

(71) Applicant: Nikolas Carmelo Marulis, Lighthouse Point, FL (US)

(72) Inventor: Nikolas Carmelo Marulis, Lighthouse Point, FL (US)

(21) Appl. No.: 19/181,920

(22) Filed: Apr. 17, 2025

Related U.S. Application Data
(60) Provisional application No. 63/712,610, filed on Oct. 28, 2024.

(51) Int. Cl. A61K 36/06; A61K 9/00; A61K 9/08; A61K 31/135; A61K 31/198; A61K 31/375; A61K 31/593; A61K 31/714; A61K 33/06; A61K 38/25; A61K 38/27; A61K 38/30

(52) U.S. Cl. CPC A61K 36/06; A61K 9/0019; A61K 9/0056; A61K 9/08; A61K 31/135; A61K 31/198; A61K 31/375; A61K 31/593; A61K 31/714; A61K 33/06; A61K 38/25; A61K 38/27; A61K 38/30

(57) ABSTRACT

The present invention relates to a customizable injectable solution comprising Human Growth Hormone (HGH), Insulin-like Growth Factor 1 (IGF-1), or a growth hormone secretagogue such as sermorelin or ipamorelin, combined with a blend of nutrients including amino acids, vitamins, electrolytes, functional mushroom extracts, and optionally mushroom mycelium. The solution is delivered via a dual-chamber syringe to preserve component stability, with mixing occurring immediately before administration. The formulation is suitable for subcutaneous, intramuscular, or intravenous injection and may be tailored for muscle growth, cognitive performance, immune support, or neuroregeneration. The invention also contemplates injectable formulations of functional mushrooms alone, as well as alternative delivery methods such as sublingual troches containing GH-inducing peptides, functional mushrooms, and ketamine. This system provides a versatile, bioavailable, and clinically adaptable approach to personalized health optimization.

This application claims the benefit of U.S. Provisional Patent Application No. 63/712,610 filed on Oct. 28, 2024, which is incorporated by reference herein in its entirety.

### FIELD OF THE INVENTION

The present invention relates to the field of nutritional and therapeutic supplements, and more specifically to customizable injectable and sublingual compositions comprising nutrients, functional mushroom extracts, mushroom mycelium, growth hormone agents such as Human Growth Hormone (HGH), Insulin-like Growth Factor 1 (IGF-1), and growth hormone secretagogues. The invention further relates to solutions formulated for parenteral or buccal administration to support a range of health outcomes, including muscle development, cognitive enhancement, immune modulation, neuroregeneration, and mood support.

### BACKGROUND OF THE INVENTION

In recent years, injectable nutrient solutions have gained widespread attention as an alternative to traditional oral supplementation. Unlike capsules or tablets that rely on gastrointestinal absorption, injectable formulations introduce nutrients directly into the bloodstream, bypassing the digestive tract entirely. This approach offers a number of advantages, particularly with respect to bioavailability, absorption rates, and speed of therapeutic effect. Many nutrients, including certain vitamins and amino acids, are known to undergo significant degradation or transformation in the acidic environment of the stomach or during hepatic first-pass metabolism, resulting in diminished efficacy when delivered orally. Consequently, intravenous, intramuscular, and subcutaneous delivery methods have emerged as viable options for optimizing nutrient uptake, particularly for users with compromised digestion, absorption issues, or specific therapeutic goals requiring rapid onset of action.

A wide variety of injectable formulations have been developed to meet the increasing demand for nutrient-based therapies. These formulations often incorporate combinations of vitamins, minerals, amino acids, and other micronutrients aimed at promoting general wellness, correcting deficiencies, and enhancing performance or recovery. In clinical settings, injectable vitamin B12 is frequently used to treat pernicious anemia and support energy metabolism, while injectable vitamin D3 is administered to address hypovitaminosis D. Athletes and individuals focused on fitness have also adopted nutrient injections to support muscle recovery, hydration, and cellular repair, often using amino acids like glutamine or branched-chain amino acids (BCAAs). Additionally, combinations of electrolytes and antioxidants have been employed to reduce oxidative stress and promote resilience to physical exertion, fatigue, or illness.

Alongside injectable nutrients, peptide hormones such as Human Growth Hormone (HGH) and Insulin-like Growth Factor 1 (IGF-1) have been widely studied and utilized for their regenerative and anabolic properties. HGH is produced naturally by the pituitary gland and is essential for growth, tissue repair, metabolism, and immune function. Recombinant HGH has been used therapeutically for growth hormone deficiency, Turner syndrome, cachexia, and other endocrine disorders. IGF-1, a downstream mediator of HGH activity, also promotes cell proliferation, muscle regeneration, and neural development, and has been used to treat growth failure and neurodegenerative conditions. Due to their potent physiological effects, both hormones are of interest not only in traditional medical contexts but also in off-label uses aimed at performance enhancement, anti-aging, and cognitive support.

However, the use of HGH and IGF-1 outside medically supervised contexts has raised significant regulatory and safety concerns. These hormones can exert wide-ranging effects across multiple organ systems, and improper use may lead to adverse events such as edema, insulin resistance, joint pain, or unintended tissue growth. There are also ethical considerations surrounding their use for enhancement rather than treatment, especially in competitive sports. Moreover, maintaining the stability and bioactivity of peptide hormones poses a notable formulation challenge. Both HGH and IGF-1 are sensitive to heat, light, pH, and enzymatic degradation, and may lose efficacy if exposed to suboptimal conditions during manufacturing, storage, or administration. As such, ensuring the structural integrity of these hormones until the point of injection is essential for safe and effective use.

In the related art, there exist several injectable compositions that attempt to combine HGH or IGF-1 with supportive nutrients in a single formulation. These products are intended to amplify the effects of the hormones by pairing them with amino acids, vitamins, and minerals known to influence metabolism, recovery, or anabolism. However, many of these formulations are limited by poor component stability, lack of customization, and undesirable interactions between ingredients. For example, certain amino acids may alter the pH or ionic strength of the solution, destabilizing the hormone. Similarly, some vitamins may act as pro-oxidants under specific conditions, leading to degradation or aggregation of sensitive proteins like HGH or IGF-1. As a result, the co-formulation of peptide hormones with nutrients remains an area of active development with substantial technical barriers.

Another problem observed in current solutions is the lack of flexibility with respect to personalization. Many commercially available products are offered as pre-mixed solutions with fixed concentrations and ingredient profiles. This limits the ability of clinicians or end users to tailor treatments to individual needs, such as optimizing dosing for different age groups, physiological states, or health goals. A one-size-fits-all approach may also increase the risk of side effects, particularly in users with allergies, metabolic conditions, or drug-nutrient interactions. Additionally, pre-mixed formulations typically require preservatives or stabilizers that may further complicate the compatibility of the active components. The ability to provide individualized nutrient-hormone ratios while maintaining pharmaceutical quality and stability is therefore an unmet need in the space.

The potential for adverse interactions between included nutrients and active hormone agents presents another serious concern. Certain amino acids may compete for cellular uptake or metabolic processing, reducing the bioefficacy of the formulation. For example, high levels of one amino acid may inhibit the absorption or transport of others, resulting in imbalanced effects or unintended metabolic consequences. Moreover, the pharmacodynamics of hormone signaling pathways can be influenced by the presence of cofactors or modulators, potentially leading to unpredictable results. Without precise control over composition, sequence of administration, and bioavailability, users may fail to achieve the desired therapeutic response or may experience unwanted effects.

Some prior solutions have attempted to mitigate these problems by separating active ingredients into different syringes or injection events. However, this approach is inconvenient and prone to dosing errors or non-compliance. A more promising method involves the use of dual-chamber syringe systems that keep incompatible components isolated until the moment of injection. This allows for real-time reconstitution, minimizing the risk of degradation or premature interaction between sensitive ingredients. Despite the advantages of such systems, existing products still tend to focus on narrow applications—such as vitamin or hormone replacement—and fail to capitalize on the full potential of combining synergistic components like nootropics, adaptogens, and functional mushrooms.

Functional mushrooms, including Lion's Mane, Reishi, Cordyceps, Chaga, Turkey Tail, and Shiitake, have gained attention for their broad health benefits and bioactive compounds. These mushrooms are rich in polysaccharides, beta-glucans, terpenes, and antioxidants, which have been shown to support immune modulation, neurogenesis, stress resilience, and mitochondrial function. However, their use in injectable formulations is extremely limited, likely due to concerns around solubility, extract standardization, and regulatory classification. Furthermore, most functional mushroom products are consumed orally, where absorption is incomplete and subject to significant variability due to digestive enzymes and gastric pH. The development of parenteral mushroom-based therapies could significantly improve the bioavailability and onset of these compounds, especially in acute or high-performance contexts.

In addition, mushroom mycelium—the root-like network of fungal cells that grows underground—contains unique compounds not found in the fruiting body and may offer different or complementary biological effects. The ability to isolate and inject mycelium-derived bioactives would broaden the therapeutic scope of mushroom-based interventions. A solution capable of incorporating either fruiting body or mycelium extracts, or both, could therefore allow for greater formulation flexibility and wider applicability. Despite this potential, few-if any-injectable products on the market utilize mushroom extracts in this way, presenting a significant opportunity for innovation and intellectual property protection.

In light of these considerations, a need exists for an improved injectable formulation that combines a growth hormone agent (e.g., HGH, IGF-1, sermorelin, ipamorelin) with a tailored blend of nutrients and functional mushroom extracts. The formulation should be capable of maintaining the chemical and structural stability of the components until administration, with minimal risk of degradation or inactivation. The ability to store the components separately—such as in a dual-chamber syringe—and mix them immediately prior to use is desirable. The nutrient blend should be customizable to include amino acids, vitamins, electrolytes, and functional mushrooms or mycelium, in ratios selected for specific therapeutic objectives. These might include muscle growth, recovery, cognition, immune modulation, or neuroregeneration.

In another desirable configuration, the injectable formulation may further incorporate nootropic agents or adaptogens to support mental performance, stress resilience, or mood regulation. Examples include ketamine, racetams, and modafinil, which may act synergistically with the neuroactive properties of Lion's Mane or other mushroom extracts. Moreover, in certain cases it may be advantageous to deliver the formulation via sublingual administration, such as in the form of a troche or lozenge. This route bypasses gastrointestinal degradation and hepatic first-pass metabolism, while offering patient convenience and improved compliance. Such a troche may include a combination of GH-inducing peptides, mushroom extracts, and nootropics, tailored for fast-acting, non-invasive cognitive support.

Accordingly, the desired solution would provide a highly modular, user-specific platform for combining growth hormone agents with functional nutrients and biologically active mushroom compounds. It should offer flexibility in formulation, stability in storage, and broad utility across multiple modes of administration. The platform would improve safety, efficacy, and convenience for users seeking enhanced physiological or cognitive performance, recovery, or general wellness. By addressing the limitations of prior art, this invention advances the field of injectable nutrient and hormone therapy into new domains of personalization, integration, and multi-pathway optimization.

### SUMMARY OF THE INVENTION

The present invention relates to an injectable solution comprising a combination of a growth hormone agent—such as Human Growth Hormone (HGH), Insulin-like Growth Factor 1 (IGF-1), or a growth hormone secretagogue including but not limited to sermorelin or ipamorelin—combined with a tailored blend of essential nutrients. The essential nutrients may include vitamins, amino acids, electrolytes, functional mushroom extracts, and optionally mushroom mycelium. The solution is formulated as a two-part system, where the primary components are stored separately and combined immediately before administration to maintain stability and maximize the bioavailability of the active ingredients. The system is customizable, allowing for variation in the hormone agent, nutrient blend, concentration, and dosing, depending on individual health needs.

The invention offers several improvements over prior art formulations. The two-part delivery system protects labile components such as peptide-based hormones from degradation, ensuring full potency at the time of use. The customizable composition enables precision targeting of health outcomes, including but not limited to muscle growth, immune support, and cognitive enhancement. Functional mushrooms and their mycelium are incorporated to provide synergistic effects not traditionally offered in injectable nutrient formulations. These may include neuroregenerative, anti-inflammatory, immunomodulatory, and mood-supporting benefits. The ability to separately include mushroom fruiting bodies and mycelial extracts expands therapeutic scope, as different components can exert different bioactive effects. Additionally, the solution is suitable for subcutaneous, intramuscular, or intravenous injection, providing flexibility in delivery route depending on clinical goals. The formulation may optionally include nootropic agents such as ketamine, racetams, or modafinil. In another aspect, sublingual formulations such as troches or lozenges may be used to deliver a combination of functional mushrooms, GH-inducing peptides, and ketamine via buccal absorption.

In a first implementation of the invention, an injectable solution comprises:
a first component comprising a growth hormone agent selected from the group consisting of Human Growth Hormone (HGH), Insulin-like Growth Factor 1 (IGF-1), sermorelin, ipamorelin, and other growth hormone secretagogues;
a second component comprising a nutrient blend, the nutrient blend including at least one ingredient selected from the group consisting of amino acids, vitamins, electrolytes, functional mushroom extracts, and mushroom mycelium; wherein
the first and second components are stored separately and mixed immediately prior to administration.

In another aspect, the first and second components may be stored in a dual-chamber syringe.

In another aspect, the functional mushroom extracts may be selected from the group consisting of Lion's Mane (Hericium erinaceus), Reishi (Ganoderma lucidum), Cordyceps (Cordyceps militaris), Chaga (Inonotus obliquus), Turkey Tail (Trametes versicolor), and Shiitake (Lentinula edodes).

In another aspect, the mushroom component may include an isolated mycelium extract separated from the fruiting body.

In another aspect, the amino acids may include one or more of leucine, isoleucine, valine, glutamine, or creatine.

In another aspect, the vitamins may include one or more of niacin, vitamin D3, vitamin B12, or vitamin C.

In another aspect, the electrolytes may include one or more of sodium, potassium, magnesium, or calcium.

In another aspect, the solution may further include a stabilizing agent selected from the group consisting of trehalose, mannitol, or sorbitol.

In another aspect, the solution may further include a preservative selected from the group consisting of benzyl alcohol, chlorobutanol, or methylparaben.

In another aspect, the growth hormone agent may be present in a concentration between 0.25 to 6 International Units (IU) per mL.

In another aspect, the nutrient blend may be tailored for one or more targeted outcomes selected from the group consisting of muscle growth, cognitive enhancement, immune support, or stress resilience.

In another aspect, the combined solution may be formulated for subcutaneous administration.

In another aspect, the combined solution may be formulated for intramuscular administration.

In another aspect, the combined solution may be formulated for intravenous administration.

In another aspect, the solution may further include a nootropic agent selected from the group consisting of racetams, modafinil, or ketamine.

In another aspect, the growth hormone agent may be sermorelin or ipamorelin, and the nutrient blend may include Lion's Mane and vitamin B12.

In another aspect, the solution may be administered according to a schedule selected from the group consisting of daily, weekly, or cyclic administration.

In another implementation of the invention, an injectable solution comprises:
a functional mushroom extract and optionally a mushroom mycelium extract; wherein
the composition is free of Human Growth Hormone (HGH), Insulin-like Growth Factor 1 (IGF-1), and any growth hormone secretagogue; and wherein
the composition is formulated for parenteral administration and configured to promote cognitive enhancement or immune support.

In another implementation of the invention, an injectable solution comprises:
a growth hormone secretagogue selected from the group consisting of sermorelin, ipamorelin, and GHRP-6; and
a functional mushroom extract selected from the group consisting of Lion's Mane, Reishi, or Chaga; and ketamine; wherein
the formulation is administered as a troche or lozenge for buccal absorption.

In another aspect, the sublingual formulation may be tailored for cognitive performance, mood enhancement, or neuroplasticity support.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a perspective view of the dual-chamber syringe, illustrating its design with two separate chambers, one containing the Human Growth Hormone (HGH) or Insulin-like Growth Factor 1 (IGF-1) and the other containing a nutrient blend, in accordance with one embodiment of the invention;

FIG. 2 presents a detailed flowchart of the step-by-step process for preparing and administering the injectable solution, showing the sequential steps involved in mixing the components within the syringe, selecting the injection site, and administering the solution;

FIG. 3 presents a perspective view of a chart listing the possible compositions of the nutrient blend, illustrating various combinations of amino acids, vitamins, electrolytes, and mushroom extracts, and their potential benefits when combined with HGH or IGF-1; and

FIG. 4 presents a diagrammatic view of the human body, highlighting the recommended injection sites for subcutaneous and intramuscular administration of the injectable solution, indicating optimal locations for delivering the solution for maximum efficacy.

Like reference numerals refer to like parts throughout the several views of the drawings.

### DETAILED DESCRIPTION

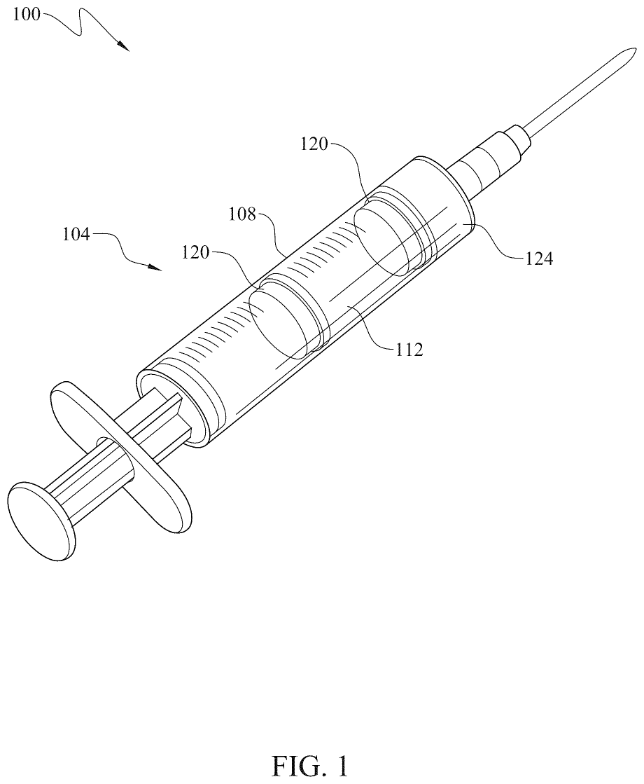

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

The present invention, referenced as 100, introduces a comprehensive health-optimization system comprising an injectable solution designed to combine Human Growth Hormone (HGH) or Insulin-like Growth Factor 1 (IGF-1), or any suitable growth hormone secretagogue such as sermorelin, ipamorelin, or related peptides, with a customizable blend of nutrients. The formulation is intended to support various physiological and cognitive functions including but not limited to anabolic support, recovery acceleration, immune function, and neurological enhancement. This solution is delivered via a dual-chamber syringe 104, strategically configured to ensure the stability and integrity of the individual components until the moment of administration. By maintaining ingredient separation, the system prevents degradation of labile compounds and facilitates optimal bioactivity upon injection. In some embodiments, the invention further encompasses injectable formulations comprising functional mushroom extracts alone, without HGH or GH secretagogues, to target users seeking neurocognitive or immunomodulatory benefits without hormonal supplementation. The formulation platform is intended to be flexible and scalable to support a wide range of user-specific wellness objectives, clinical applications, or performance-enhancement protocols.

The dual-chamber syringe 104, as illustrated in FIG. 1, comprises two physically separated chambers—108 and 112—engineered to house mutually reactive or stability-sensitive ingredients. Chamber 108 may contain HGH, IGF-1, or a peptide-based growth hormone secretagogue such as sermorelin, ipamorelin, or CJC-1295. These compounds are often temperature-sensitive and biologically fragile, necessitating isolation from other active agents or excipients prior to use. Chamber 112 is reserved for the nutrient solution, which may include combinations of amino acids, vitamins, electrolytes, and functional mushroom extracts. The mushroom constituents may be delivered as extracts from the fruiting body or the mycelium, with each exhibiting distinct pharmacodynamic properties. Functional mushrooms such as Lion's Mane, Reishi, Cordyceps, Chaga, Turkey Tail, and Shiitake are included for their well-documented benefits on cognitive function, neurogenesis, immune response modulation, and stress adaptation. The separation of mycelium from the fruiting body allows for precise formulation and enables inclusion of compounds with potentially superior bioavailability and therapeutic specificity.

The nutrient blend stored in chamber 112 is designed to be fully customizable and adaptable for various clinical, athletic, and general wellness applications. The amino acid components may include leucine, isoleucine, valine, glutamine, and creatine, each selected for their specific contributions to muscle protein synthesis, ATP regeneration, nitrogen retention, and post-exercise recovery. Vitamins such as niacin, vitamin D3, vitamin B12, and vitamin C provide essential cofactors for metabolic processes, immune modulation, and neurotransmitter synthesis. Electrolytes such as sodium, potassium, magnesium, and calcium play critical roles in cellular signaling, hydration balance, and cardiovascular function. The inclusion of mushroom extracts-both from the fruiting body and the mycelium-introduces adaptogenic and neuroprotective effects that may synergize with the hormonal or peptide base. The blend may be tailored to individual biomarker profiles, dietary gaps, or performance targets, offering a personalized approach to injectable nutrient therapy. Each ingredient is selected and formulated to remain chemically stable during short-term storage and to function in concert when mixed prior to administration.

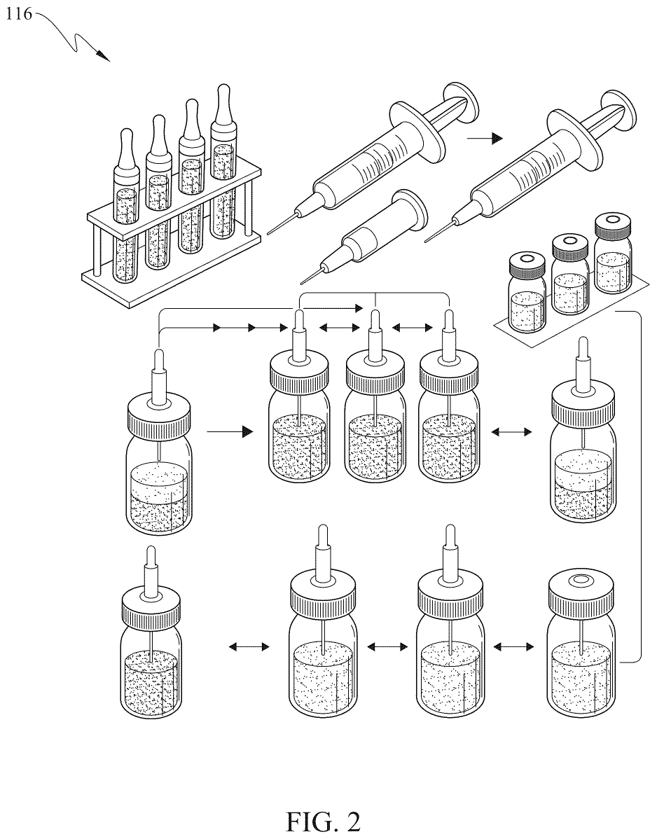

FIG. 2 illustrates the operational procedure 116 for preparing and administering the injectable solution. The first step involves selection of the active compound dosage to be stored in chamber 108—this may be HGH, IGF-1, or a growth hormone secretagogue, selected based on the user's age, metabolic rate, and desired physiological outcomes. The dosage range for these compounds may vary from as low as 0.25 IU to as high as 6 IU per mL, allowing for both conservative and aggressive administration protocols. In parallel, the nutrient solution is formulated and loaded into chamber 112, based on the user's intended goals such as muscle recovery, neurological performance, or general vitality. The syringe is then primed with a mixing mechanism 120, which could include a dual-plunger system, breakable membrane, or mechanical agitator to ensure complete blending of the solutions immediately before injection. The act of mixing ensures that sensitive compounds are not subjected to prolonged contact that could reduce their potency or lead to chemical interactions that alter efficacy. This pre-injection mixing step is critical for preserving pharmacological integrity and for enabling a synergistic response upon delivery.

Following mixing, the resulting solution is drawn into a single injection-ready chamber 124 within the syringe. The delivery route may be selected based on clinical or personal preference, pharmacokinetic profiles, or target therapeutic outcomes. Subcutaneous injection may be ideal for steady-state absorption, such as in the case of daily microdosing protocols. Intramuscular injection is suitable for larger volumes or for compounds requiring more rapid systemic uptake. Intravenous administration, which has now been included as a preferred route in some embodiments, allows for immediate systemic distribution and maximal bioavailability, particularly for clinical use or under professional supervision. The choice of administration route may also be guided by the molecular weight, stability, and dispersion characteristics of the formulation. The flexibility to use any of the three routes allows the invention to accommodate various settings, from in-clinic administration to at-home wellness use under prescription.

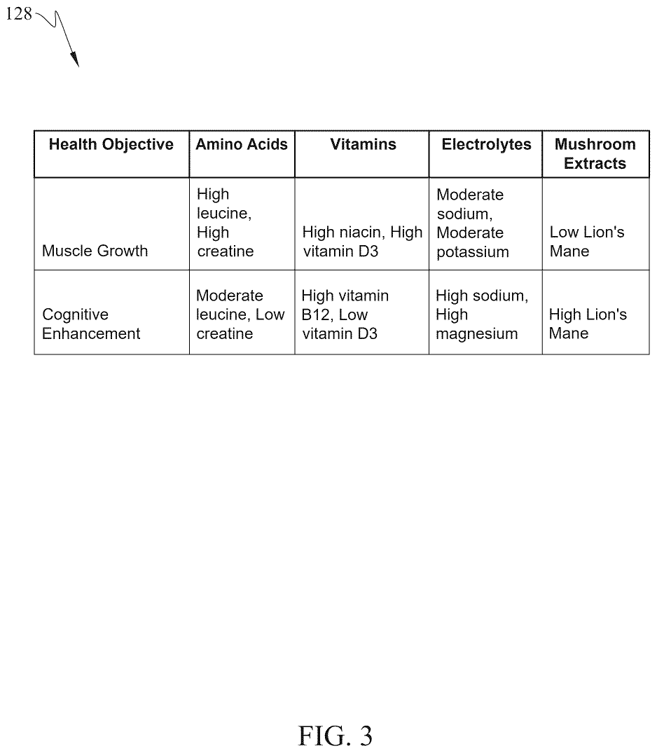

FIG. 3 presents a chart 128 which provides a reference for possible combinations and concentrations of nutrients and mushroom extracts tailored to specific goals. For instance, an anabolic-focused blend may incorporate elevated levels of leucine and creatine, complemented by vitamin D3 and magnesium for skeletal support. A cognitive-focused blend may emphasize Lion's Mane, vitamin B12, Reishi mycelium, and acetyl-L-carnitine for enhanced memory, focus, and mood regulation. Immune-boosting formulas may draw upon Chaga, Turkey Tail, vitamin C, and zinc to support T-cell function and antioxidant defenses. The chart may also list optional enhancements such as resveratrol, curcumin, or omega-3 fatty acids for additional therapeutic layering. Each blend is a modular template, allowing clinicians or trained users to build custom formulations suited to short-term cycles or long-term health protocols. The chart thus serves as a tool for both prescriptive formulation and exploratory iteration.

This level of blend modularity offers unprecedented flexibility and user customization, a core objective of the invention. The modular system supports dynamic responses to lifestyle shifts, training phases, illness recovery, or preventative care protocols. For example, an athlete preparing for competition may shift to a high-creatine, high-electrolyte blend, while someone recovering from illness may favor higher mushroom content with anti-inflammatory support. The composition may also be periodically reassessed using biomarker tracking or diagnostic panels to optimize ongoing administration. This customization capability enhances not only the user experience but also the clinical utility and market differentiation of the invention. The nutrient blend may also evolve based on scientific advancements or feedback from ongoing usage. In sum, the invention supports personalized medicine within an injectable framework.

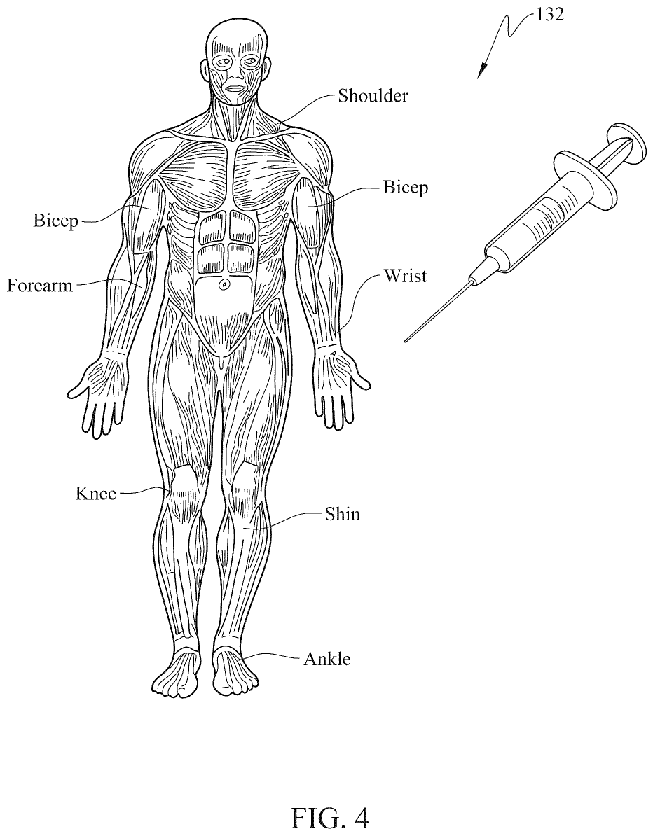

FIG. 4 depicts the preferred anatomical sites 132 for injection based on the selected administration route. For subcutaneous injections, typical sites may include the abdomen (2 inches from the navel), anterior thighs, or outer upper arms. Intramuscular sites include the deltoid (upper arm), vastus lateralis (thigh), and gluteus maximus (buttocks), chosen for their robust muscle mass and safe depth access. For intravenous administration, the cephalic, basilic, or median cubital veins in the forearm are commonly used, often requiring venipuncture technique. The selection of site may be informed by user comfort, rotational protocol, and compound-specific absorption patterns. FIG. 4 may illustrate these sites with indicators corresponding to the injection type, facilitating both training and application. Safety considerations such as needle gauge, injection angle, and aseptic technique are assumed as part of standard practice.

The method of injection may vary according to the final formulation and the desired pharmacodynamic profile. The user or practitioner begins by selecting the appropriate injection site, followed by aseptic skin preparation. Once the dual-chamber syringe 104 is activated, the components are mixed and loaded into the single chamber 124. The needle is then inserted at the designated angle and depth, depending on the route—45 degrees for subcutaneous, 90 degrees for intramuscular, and direct vein access for intravenous. The injection is administered slowly to minimize discomfort and ensure even distribution. Post-injection protocols may include applying pressure with sterile gauze and massaging the site to aid dispersion.

Optional components may include nootropic compounds such as racetams, modafinil, or ketamine. Ketamine, in particular, is of interest due to its known neuroplastic and antidepressant properties and is already used in sublingual and injectable forms. Its synergistic use with Lion's Mane and growth hormone secretagogues could offer enhanced effects on mood regulation, memory, and cognitive flexibility. Sublingual variations may include troches that deliver combinations of GH peptides, mushroom extracts, and ketamine, bypassing hepatic metabolism and enabling rapid onset. These sublingual formats can be formulated with binders and flavoring agents for user compliance and absorption efficacy. Including these options broadens the potential use cases for the invention beyond purely injectable applications.

To maintain compound stability, the formulation may include stabilizing agents such as trehalose, mannitol, or sorbitol. These excipients prevent denaturation of peptides and proteins, and help regulate osmotic pressure in solution. Additionally, preservatives such as benzyl alcohol, chlorobutanol, or methylparaben may be incorporated to reduce microbial contamination risk and extend shelf life. Buffer systems may be included to maintain pH within an optimal range for both active ingredient stability and tissue compatibility. The system may be designed for room-temperature storage or refrigerated conditions depending on the actives involved. These formulation features collectively contribute to the product's viability, safety, and efficacy.

The dual-chamber syringe 104 is central to the invention's stability and usability features. By preventing premature interaction between sensitive compounds, it avoids chemical degradation and preserves bioactivity. The design may include a snap-seal, plunger divider, or integrated mixer that facilitates in-syringe reconstitution. In more advanced embodiments, the syringe could feature a dual-barrel system that supports sequential injections or controlled-release options. Materials used in the syringe construction may include medical-grade polymers, glass, or barrier-lined plastics that prevent leaching and compound reactivity. The syringe may be configured for single-use, or in certain embodiments, as part of a refillable cartridge system compatible with autoinjector devices.

Dosing of HGH, IGF-1, or GH secretagogues may vary according to treatment goals, ranging from microdose regimens (e.g., 0.25 IU) to standard therapeutic doses (e.g., 6 IU). The flexibility in dosing supports use cases ranging from daily optimization protocols to medically supervised interventions. Nutrient dosing may also be adjusted based on lab values, patient feedback, or time of day to support circadian rhythms. Tapering or loading protocols may be employed depending on the specific ingredient profile. Multiple formulations may be designed to cycle through over time, supporting periodized health optimization. The platform also allows future expansion into different peptide classes or natural compounds.

The frequency of administration may follow a fixed schedule (e.g., daily, weekly) or an adaptive schedule based on biometric feedback, therapeutic response, or physician oversight. Troche-based or sublingual variations may provide a non-invasive route for delivering sensitive compounds like GH peptides and ketamine while maintaining rapid onset. These variations are particularly advantageous for patients unable or unwilling to use injectable forms. Other alternative routes under consideration include transdermal patches and nasal sprays, though these would require additional formulation development. Regardless of format, the core innovation remains the customizable and synergistic composition. Delivery formats are chosen to maximize adherence and efficacy for the target user.

Additives may include antioxidant molecules such as glutathione or alpha-lipoic acid, adaptogens like ashwagandha or Rhodiola rosea, and anti-inflammatory agents such as boswellia or turmeric extract. These may enhance systemic resilience, reduce inflammation, and promote neuroendocrine balance. The inclusion of these agents may be tailored based on individual needs or diagnostic criteria. Co-administration of such compounds may offer synergistic effects with the core hormone and mushroom components. These agents may be delivered in the same injection or provided as adjunct formulations. Additional bioenhancers or penetration aids may also be incorporated depending on the delivery route.

This invention offers a comprehensive solution that bridges precision supplementation, hormone therapy, and novel nootropic delivery. By combining customizable blends of nutrients, functional mushrooms, peptides, and optional agents in a dual-chamber injectable or sublingual format, the system provides unmatched personalization. It supports a spectrum of applications from athletic performance and longevity enhancement to neurological recovery and immune resilience. The ability to mix and match active agents in a controlled, stable delivery system enhances both the therapeutic and commercial value of the platform. This flexibility enables the invention to evolve with emerging science and user needs. The system is designed to be modular, evidence-driven, and scalable across demographics.

The invention is not limited to the embodiments disclosed herein. Numerous modifications and enhancements may be introduced without departing from the core inventive concept. This includes integration of biomarkers for real-time feedback, digital interfaces for dosing control, or AI-generated optimization protocols. Formulation variations may address pediatric, geriatric, or gender-specific needs. Other innovations may include microfluidic mixing, compartmentalized reservoirs, or hybrid delivery systems. Accordingly, the scope of the invention is defined by the claims and is intended to cover all variations that fall within their bounds.

Since many modifications, variations, and changes in detail can be made to the described preferred embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

What is claimed is:

1. An injectable solution comprising:
a first component comprising a growth hormone agent selected from the group consisting of Human Growth Hormone (HGH), Insulin-like Growth Factor 1 (IGF-1), sermorelin, ipamorelin, and other growth hormone secretagogues;
a second component comprising a nutrient blend, the nutrient blend including at least one ingredient selected from the group consisting of amino acids, vitamins, electrolytes, functional mushroom extracts, and mushroom mycelium; wherein
the first and second components are stored separately and mixed immediately prior to administration.

2. The injectable solution of claim 1, wherein the first and second components are stored in a dual-chamber syringe.

3. The injectable solution of claim 1, wherein the functional mushroom extracts are selected from the group consisting of Lion's Mane, Reishi, Cordyceps, Chaga, Turkey Tail, and Shiitake.

4. The injectable solution of claim 1, wherein the mushroom component comprises isolated mycelium extract separated from the fruiting body.

5. The injectable solution of claim 1, wherein the amino acids comprise at least one of leucine, isoleucine, valine, glutamine, or creatine.

6. The injectable solution of claim 1, wherein the vitamins comprise at least one of niacin, vitamin D3, vitamin B12, or vitamin C.

7. The injectable solution of claim 1, wherein the electrolytes comprise at least one of sodium, potassium, magnesium, or calcium.

8. The injectable solution of claim 1, further comprising a stabilizing agent selected from the group consisting of trehalose, mannitol, and sorbitol.

9. The injectable solution of claim 1, further comprising a preservative selected from the group consisting of benzyl alcohol, chlorobutanol, and methylparaben.

10. The injectable solution of claim 1, wherein the growth hormone agent is present in a concentration between 0.25 to 6 International Units (IU) per mL.

11. The injectable solution of claim 1, wherein the nutrient blend is tailored for one or more targeted outcomes selected from the group consisting of muscle growth, cognitive enhancement, immune support, or stress resilience.

12. The injectable solution of claim 1, wherein the combined solution is formulated for subcutaneous administration.

13. The injectable solution of claim 1, wherein the combined solution is formulated for intramuscular administration.

14. The injectable solution of claim 1, wherein the combined solution is formulated for intravenous administration.

15. The injectable solution of claim 1, further comprising a nootropic agent selected from the group consisting of racetams, modafinil, or ketamine.

16. The injectable solution of claim 1, wherein the growth hormone agent is sermorelin or ipamorelin and the nutrient blend comprises Lion's Mane and vitamin B12.

17. The injectable solution of claim 1, wherein the solution is administered according to a schedule selected from the group consisting of daily, weekly, or cyclic administration.

18. An injectable solution comprising:
a functional mushroom extract and optionally a mushroom mycelium extract; wherein
the composition is free of Human Growth Hormone (HGH), Insulin-like Growth Factor 1 (IGF-1), and any growth hormone secretagogue; and wherein
the composition is formulated for parenteral administration and configured to promote cognitive enhancement or immune support.

19. A sublingual formulation comprising:
a functional mushroom extract and optionally a mushroom mycelium extract; wherein
the composition is free of Human Growth Hormone (HGH), Insulin-like Growth Factor 1 (IGF-1), and any growth hormone secretagogue; and wherein
the composition is formulated for parenteral administration and configured to promote cognitive enhancement or immune support.
a growth hormone secretagogue selected from the group consisting of sermorelin, ipamorelin, and GHRP-6; and
a functional mushroom extract selected from the group consisting of Lion's Mane, Reishi, or Chaga; and ketamine; wherein
the formulation is administered as a troche or lozenge for buccal absorption.

20. The sublingual formulation of claim 19, wherein the formulation is tailored for cognitive performance, mood enhancement, or neuroplasticity support.
